(19) United States
(12) Patent Application Publication
(10) Pub. No.: US 2024/0157105 A1
(43) Pub. Date: May 16, 2024

(54) TATTOO INKING SYSTEM

(71) Applicant: Scott Michael BARRY, Saugus, MA (US)

(72) Inventor: Scott Michael BARRY, Saugus, MA (US)

(21) Appl. No.: 18/506,314

(22) Filed: Nov. 10, 2023

Related U.S. Application Data
(60) Provisional application No. 63/383,122, filed on Nov. 10, 2022.

(51) Int. Cl. A61M 37/00; A61M 39/24

(52) U.S. Cl. CPC A61M 37/0084; A61M 39/24; A61M 2205/075; A61M 2205/121

(57) ABSTRACT

Methods of tattooing and tattoo inking systems comprising a housing, an ink reservoir, a pump and a tattoo cartridge. According to a further embodiment the tattoo inking system comprises a system of channels and valves that allow the artist to provide ink to the tattoo cartridge while working and without dipping.

### BACKGROUND

The process and apparatus used for tattooing has remained largely unchanged for many decades. One step in the process involves repeatedly dipping a tip of the tattoo machine into an ink well to load an ink reservoir with a small amount of ink to perform a tattooing.

### SUMMARY

In one aspect, a tattoo inking system is provided, the system including a housing having a proximal end and a distal end, an ink reservoir in the proximal end, a tattoo cartridge mounted on the distal end, a channel providing fluid communication between the ink reservoir and the tattoo cartridge, and a pump in communication with the channel. The system can include a mechanical source for reciprocating the needle, and the system can be mechanically connected to a housing including a rotary or coil machine.

The tattoo inking system can include a pump that is configured to draw ink from the ink reservoir and deposit the into the tattoo cartridge. It can include a one-way valve between the ink reservoir and the pump and/or a one-way valve between the pump and the outlet to the tattoo cartridge. The pump can be mounted on an upper surface of the housing. A portion of the channel leading from the ink reservoir can be on a lower side of the ink reservoir opposite the upper surface of the housing while the pump is in the upper portion of the housing. A portion of the channel between the pump and the tattoo cartridge can comprise a flexible trunk and the flexible trunk is constructed and arranged to provide fluid communication through an orifice in a tattoo cartridge. In another embodiment, the tattoo cartridge is fixed to the housing and a portion of the channel from the pump to the tattoo cartridge is an annular passageway. The pump can be a compressible bulb and the one-way valve between the ink reservoir and the pump can be made from a flexible polymer. The one-way gasket can be an annular gasket and can serve as both a valve and a seal. The housing can be cylindrical, can be of metal or polymer, can include an outer layer of polymer for gripping and can include a design around the circumference to aid with gripping.

In another aspect, a method of applying ink to a tattoo needle is provided, the method comprising grasping a tattoo machine housing, activating a pump mounted on the housing to draw ink from a reservoir in the housing, pumping the ink into a tattoo cartridge, and contacting the tattoo needle with the ink. Drawing the ink from the reservoir can include opening a one-way valve. The valve can be a passive valve. Pushing the ink into the tattoo cartridge can close the one-way valve upstream of the pump and open a second one-way valve downstream of the pump. The ink can be flowed through a flexible tube into an orifice in the tattoo cartridge or it can be flowed into a cavity defined by a wall of the tattoo cartridge. The cavity can be an annular cavity formed between an inner wall of the cartridge and an outer wall of a cartridge support. The method can include removing a cap from the reservoir, adding ink to the reservoir and replacing the cap. The cap can be sealed by screwing it onto the grip, snapping it onto the grip, or clamping it onto the grip. The method can also include steps that are known in the art, such as activating a motor, changing a needle and reciprocating the tattoo needle to deposit ink into the dermis of a human.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is profile view of one embodiment of a tattooing device;

FIG. 2 is a sectional view of one embodiment of a tattooing device;

FIG. 3 is a is a sectional view of a portion of another embodiment;

FIG. 4A is a sectional view of the embodiment of FIG. 3;

FIG. 4B is a close-up view of one portion of FIG. 4A; and

FIG. 4C is a close-up view of another portion of FIG. 4A.

### DETAILED DESCRIPTION

The present disclosure will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described.

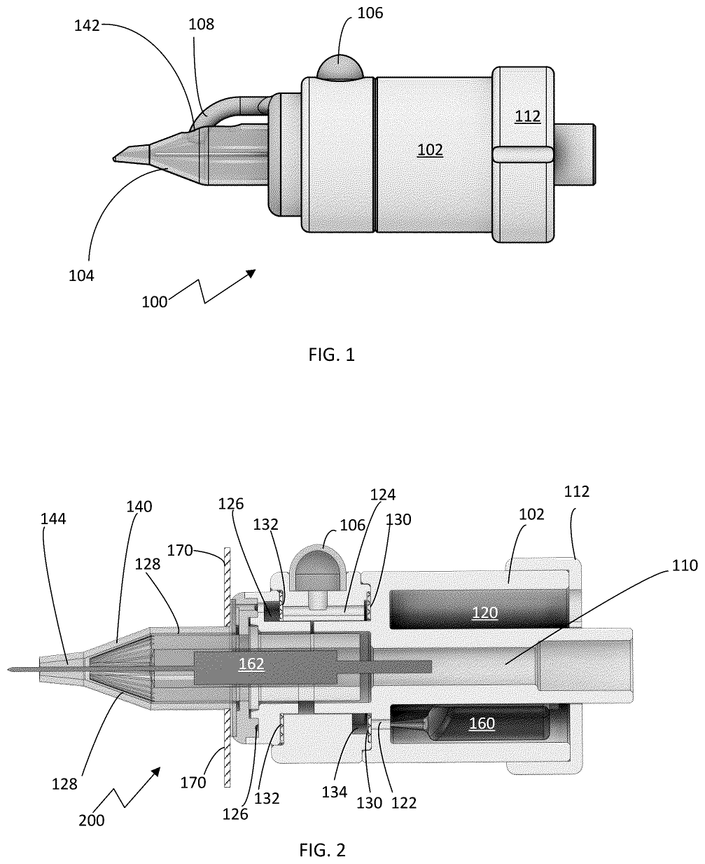

The term “at least” followed by a number is used herein to denote the start of a range beginning with that number (which may be a range having an upper limit or no upper limit, depending on the variable being defined). For example, “at least 1” means 1 or more than 1. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The term “and/or” in a claim means that the claim includes any one of the limitations preceding the term, the limitation following the term, or any combination of the limitations. Where spatial directions are given, for example above, below, top, and bottom, such directions refer to the tattoo inking system as represented in FIG. 1, unless identified otherwise.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated.

The term “substantially” means that the property is within 90% of its desired value.

The present disclosure relates to a method of tattooing and a tattoo inking device that allows the artist to provide ink to the needle on demand without dipping. It can be used with any size or type of needle available to those of skill in the art, such as liners and shaders. The device can comprise a grip, an ink reservoir in the grip, a pump, and a channel system for providing fluid communication between the reservoir and the tip.

In one aspect a device is provided that includes a tattoo grip (housing), an ink reservoir, a pump and an ink outlet. The device includes an axial bore therethrough so that it can be used with conventional tattoo machines including coil machines and rotary machines. The bore may vary in diameter at different portions of the device. The device can include an internal ink reservoir that is situated inside the grip of the housing. In some embodiments ink is poured and stored directly in the reservoir. In other embodiments, an ampule containing ink can be placed in the reservoir and fluidly connected to the device. The reservoir can be annular shaped with the axial bore passing through the center of the annulus. Although the reservoir can be in the shape of a complete circle it can also be a portion of a circle, such as a half or third circle. The volume of the reservoir can be, for example, greater than 1 mL, greater than 5 mL, greater than 10 mL, less than 20 mL, less than 10 mL or less than 5 mL. The reservoir can be filled via a removable cap, a septum or a valve.

The device can also include a pump that moves ink from the reservoir to the cartridge or tip of the needle. The pump can provide vacuum that draws ink from the ink reservoir and can provide positive pressure that flows ink into a tattoo cartridge where it contacts that needle tips. The pump can be manually powered and does not need an external source of power, such as electricity, hydraulics or pneumatics. In some embodiments, the pump can be operated by the same hand that is holding the tattoo machine, for instance with a finger or thumb of the operating hand. The pump can be a flexible bulb that decreases in volume when pressed. The decrease in volume moves ink toward the distal end of the device. When the bulb is released, suction is applied, and ink is drawn in from the reservoir. The bulb can be of polymeric material and can be transparent.

The device includes a fluid channel that provides a pathway from the ink reservoir at the proximal end of the device to the tattoo cartridge at the distal end. The channel can include one or more valves that can be check valves, such as one-way valves. The valves can be passive, meaning that they are not externally activated and simply respond to changes in pressure. The one-way valves can force the ink to only travel in one direction when pressure and vacuum are alternately applied. The valves can prevent backflow of ink and can be positioned to move the ink in one direction. For example, the ink can be moved from the ink reservoir to a channel in fluid communication with the pump, through a one-way valve, into the pumping chamber, through another one valve and into a channel that leads to the tattoo cartridge. The one-way valves can comprise flaps of flexible polymer that seal a passageway when pressurized and open when subjected to vacuum. The polymer can be, for example, flexible silicone. In some cases the polymer can be in the shape of an annular gasket that forms a circle around the housing. It can serve to provide a seal as well as to perform as a one-way valve.

A tattoo cartridge connectable to the distal end of the device can be a conventional disposable cartridge that includes an opening at the tip for the needle and another laterally oriented opening. The device can include a tube, such as a flexible tube, that can direct ink through the lateral opening into the tattoo cartridge where it can contact the tip of the needle. In other embodiments, the cartridge can be one that need not have a lateral opening and can be directly in fluid communication with an internal channel leading from the pump. The cartridge can form an annular space between the inside wall of the cartridge and an outer wall of the cartridge support, the outer wall of the cartridge support surrounding the needle. When installed, the distance between the inner wall of the cartridge and the outer wall of the distal portion of the housing can be consistent and can be, for example, less than 10 mm, less than 5 mm or less than 2 mm. The inner wall of the cartridge and the outer wall of the distal portion of the housing can be the same shape such as, for example, cylindrical or conical. This space can be sized to promote capillary flow while preventing excessive flow that would cause dripping.

FIG. 1 provides a profile view of one embodiment of a device 100 that includes an on-board ink storage and feeding mechanism. Except for bulb 106 and trunk 108 the device can be sized and shaped like a conventional grip. The device includes housing 102 and replaceable tattoo cartridge 104. Housing 102 can include a grip, and the two terms are used interchangeably herein. The feeding mechanism can include a pump. As shown, the pump includes a squeezable bulb 106 that can be pressed by the operator. The bulb can be dome shaped, as shown. The bulb can be oriented on the top of the grip, or housing, so that it can be easily accessed by the working hand. In other embodiments the bulb can be positioned below the surface of the grip so that it does not rise above the outer surface of the grip. As shown, the direction of movement of the bulb is orthogonal to the longitudinal axis of the device. This allows the practitioner to push downward toward the center of mass to dispense ink. Trunk 108 can be stiff or flexible and can be sized so that it is retained in orifice 142. Trunk 108 can be biased in a straight position aligned with the axis of the device so that it does not interfere with installation and removal of cartridge 104. Once a cartridge is installed, trunk 108 can be tucked into orifice 142 and can be retained there while the tattooing machine is in use. Cap 112 seals the reservoir after the reservoir has been filled.

FIG. 2 provides a cross-sectional view of device through the midline. As shown, the proximal end is to the right and distal end to the left. The direction of ink flow through the device is from proximal end to distal end. Housing 102 defines annular reservoir 120 that surrounds central bore 110. Central bore 110 houses a spring and needle shaft, as is known in the art. Reservoir 120 can completely surround the central bore 110 or can be limited to only a portion, for example, one half (180°) or one third (120°) of the radial space. When the reservoir does not include a full 360° annulus, it can be centered at the bottom portion of housing 102 so that when the device is held as shown, the ink in the reservoir settles to the bottom and can be drawn through orifice 122. Access to the reservoir can be gained by removing cap 112 that provides a fluid type seal on the proximal end of housing 102. Cap 112 can be secured by, for example, a snap fit, threads, or a clamp. A gasket or O-ring can be included to prevent leaking. For adding ink to reservoir 120, the device can be held in vertical alignment so that the proximal opening is pointed upwards. Once the cap is secured to the housing, the device can be held in any position without leaking.

Orifice 122 can be located in the lower portion of housing 102 so that it is submerged in ink when the device is held horizontally or in a forward leaning position, such as when working. The flow from right to left of ink through orifice 122 is blocked by valve 130. As shown, valve 130 is a flexible cylindrical ring of silicone polymer that circles the central axis of the device. As positioned, valve 130 opens when the pressure in ink channel 134 is less than the pressure in reservoir 120. When there is no pressure differential, or when pressure is greater in ink channel 134 than in reservoir 120, valve 130 is pressed against the internal wall of housing 102 and prevents the flow of ink through orifice 122.

Ink channel 134 is in fluid communication with channel 124. Flexible bulb 106 is in fluid communication with channel 124 so that when bulb 106 is depressed the pressure increases in channels 124 and 134 and valve 130 is closed. When bulb 106 is first depressed it expels air through the cartridge. When bulb 106 is released, the pressure decreases in channels 124 and 134, valve 130 moves to the left, and ink is free to flow from orifice 122 to channel 134 and channel 124. Part, or all, of bulb 106 may also fill with ink. When bulb 106 is depressed again, the resulting increase in pressure pushes valve 132 open and ink flow from channel 124 is expelled into annular void 126. Void 126 is in fluid communication with annular space 128 that is formed between the inner wall of cartridge 140 and the outer wall of cartridge support 146. Annular space 128 can be sized so that ink advances to tip void 144 where it can be deposited on the needle. The space can also be sized so that ink is limited to coating the needle and does not drip out of the cartridge. Cartridge 140 can be held in place by flange 170 which may in turn by kept in compression by threaded rods mechanically connected to housing 102. The stroke volume can be consistent so that the operator knows how much ink is being moved with each stroke. For example, the pump stroke volume can be 1 mL, 2 mL or 3 mL. In the example shown, the stroke volume is the difference in bulb volume between the extended state and the depressed state.

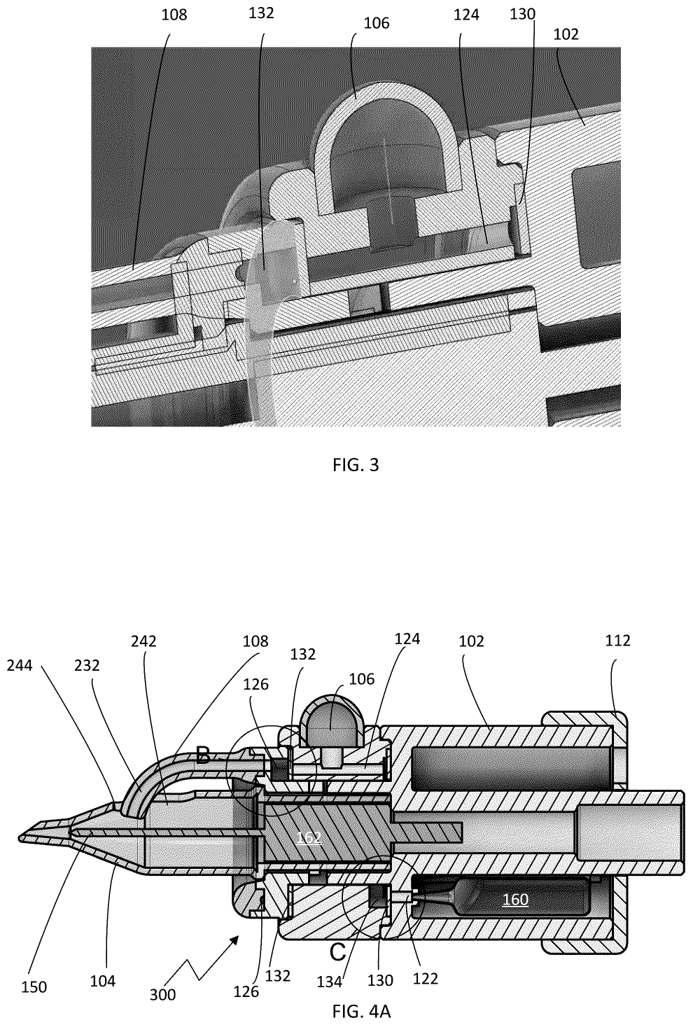

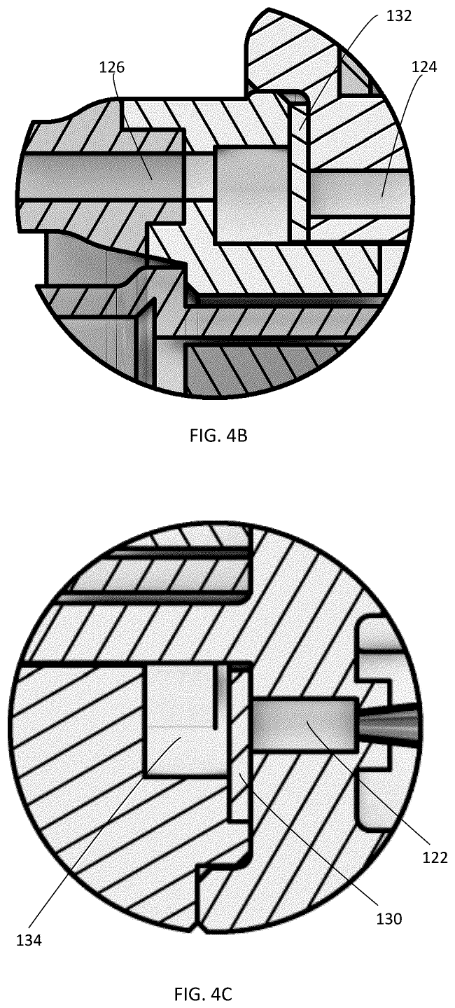

In another embodiment 300 shown in FIGS. 3, 4A, 4B and 4C. FIG. 3 shows a cutaway view of a device emphasizing a gasket, a portion of which functions as one-way valve 132. Valve 132 opens to a connector that is fluid communication with trunk 108. Trunk 108 defines lumen 232 through which ink is expressed during the increase in pressure caused by the action of the pump. Cartridge 104 can be a standard off-the-shelf cartridge available to tattoo practitioners. Cartridge 104 includes an opening 142 that provides access to the interior of the cartridge where needle 150 resides. Opening 142 is on the upper side of the cartridge (when installed) so that ink does not run out of the cartridge. The cartridge can be held in place by a snap fit, a bayonet fit or another connector that keeps it securely in place. Trunk 232 can be bent downward and tucked under lip 244. In this way, the artist can activate pump 106 to add ink to the interior of cartridge 104 without stopping work. Piston 162 is the central piston of the system.

In another embodiment ink is provided by ampule 160. Ampule 160 can be made of flexible polymer such as a polyolefin and can be disposable. The neck of ampule 160 can include a tear off cap or a screw cap and the neck of the ampule can be sized to mate with orifice 122 either by a friction fit or screw threads, for example. In this embodiment, reservoir 120 provides a housing for ampule 160 rather than directly holding the ink. As ink is drawn from ampule 160, the ampule collapses. If an ampule is removed before it is emptied, it can be re-inserted in the device for use at a later time. In some embodiments, bulb 106 is unnecessary and pumping can be achieved by compressing the ampule directly. For instance, housing 102 can include a lever, a plunger or an opening that provides mechanical access to the ampule. The volume of the ampule can be greater than 1 mL, greater than 5 mL, greater than 10 mL, less than 20 mL or less than 10 mL.

Ampule 160 may be constructed of a polymer such as a resilient polymer and/or an FDA approved polymer. A resilient polymer is one that returns to its original shape after being deformed. Examples include polyolefins such as polyethylene and polypropylene, silicone, and fluoropolymers such as PTFE. In various embodiments, the cartridge can have an aspect ratio (height to width) of greater than 1:1, greater than 2:1 greater than 3:1, less than 50:1, less than 25:1 or less than 10:1. The cartridge can be, for example, cylindrical, pillow shaped, or half-moon shaped in cross section. The grip, or housing, 102 may be constructed of a resilient polymer such as, for example, silicone or natural rubber, or of polymers, metals, metal alloys, or some combination thereof, for example. It can be molded or machined, for example. In some embodiments, the housing 102 may be constructed out of materials that are known in the art to be autoclavable. The housing can have a length of between 5.0 inches and 1.0 inch, between 4.0 inches and 1.0 inch or between 3.0 inches and 1.0 inch. It may be substantially cylindrical and can have a diameter of between 0.5 and 3.0 inches, between 1.0 and 3.0 inches or between 1.0 and 2.0 inches. Cartridge 140 may be constructed of a variety of materials such as a polymer, including thermoset and thermoformed polymers for example, and is preferably sized to allow ink to pass through when the cartridge is compressed and to not allow ink flow when the pressure in the cartridge is the same as outside the cartridge. Trunk 108 may be constructed of a variety of polymers, for example, PVC, ABS, polycarbonate, nylon, thermopolymers and urethanes. Bulb 106, and other polymeric components, can be transparent or translucent.

Cartridge support 146 can include a bore therethrough that allows for the passage and reciprocation of the drive rod and/or the needle. A portion of the cartridge support can be cylindrical and another portion can be conical. For example, a proximal portion can be cylindrical while a distal portion can be conical. The exterior surface of the conical portion can be of the same slope as the cartridge 140 to allow for the formation of void 128.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

Wherefore, I/we claim:

1. A tattoo inking system comprising:
a housing having a proximal end and a distal end;
an ink reservoir in the proximal end;
a tattoo cartridge mounted on the distal end;
a channel providing fluid communication between the ink reservoir and an outlet positioned to provide ink to a tattoo cartridge; and
a pump in communication with the channel.

2. The tattoo inking system of claim 1 wherein the pump is configured to draw ink from the ink reservoir and deposit the into the tattoo cartridge.

3. The tattoo inking system of claim 1 comprising a one-way valve between the ink reservoir and the pump.

4. The tattoo inking system of claim 1 comprising a one-way valve between the pump and the tattoo cartridge.

5. The tattoo inking system of claim 1 wherein the pump is mounted on an upper surface of the housing.

6. The tattoo inking system of claim 5 wherein a portion of the channel leading from the ink reservoir is on a lower side of the ink reservoir opposite the upper surface of the housing.

7. The tattoo inking system of claim 1 wherein the pump is in the upper portion of the housing.

8. The tattoo inking system of claim 1 wherein a portion of the channel between the pump and the tattoo cartridge comprises a flexible trunk.

9. The tattoo inking system of claim 8 wherein the flexible trunk is constructed and arranged to provide fluid communication through an orifice in the tattoo cartridge.

10. The tattoo inking system of claim 1 wherein the tattoo cartridge is fixed to the housing and a portion of the channel from the pump to the tattoo cartridge is an annular passageway.

11. The tattoo inking system of claim 1 wherein the pump comprises a compressible bulb.

12. The tattoo inking system of claim 3 wherein the one-way valve between the ink reservoir and the pump comprises a flexible polymer.

13. The tattoo inking system of claim 12 wherein the one-way valve between the ink reservoir and the pump comprises an annular gasket.

14. The tattoo inking system of claim 1 wherein the housing is cylindrical.

15. A method of applying ink to a tattoo needle, the method comprising:
grasping a tattoo machine housing;
activating a pump mounted on the housing to draw ink from a reservoir in the housing;
pumping the ink into a tattoo cartridge; and
contacting the tattoo needle with the ink.

16. The method of claim 15 wherein drawing the ink from the reservoir includes opening a one-way valve.

17. The method of claim 16 wherein pushing the ink into the tattoo cartridge comprises closing the one-way valve and opening a second one-way valve.

18. The method of claim 15 comprising flowing the ink through a flexible tube into an orifice in the tattoo cartridge.

19. The method of claim 15 comprising flowing the ink into an annular cavity defined by a wall of the tattoo cartridge.

20. The method of claim 15 further comprising reciprocating the tattoo needle to deposit ink into a dermis of a human.
